(19) United States
(12) Patent Application Publication
(10) Pub. No.: US 2015/0119977 A1
(43) Pub. Date: Apr. 30, 2015

(54) SYSTEM AND METHOD TO LIMIT CEREBRAL ISCHEMIA

(71) Applicant: THE REGENTS OF THE UNIVERSITY OF MICHIGAN, Ann Arbor, MI (US)

(72) Inventors: Juan Parodi, Buenos Aires (AR); Ramon Berguer, West Bloomfield, MI (US)

(21) Appl. No.: 14/524,711

(22) Filed: Oct. 27, 2014

Related U.S. Application Data
(60) Provisional application No. 61/897,459, filed on Oct. 30, 2013.

(51) Int. Cl. A61F 2/01; A61F 2/90; A61L 27/36; A61F 2/24

(52) U.S. Cl. CPC A61F 2/013; A61F 2/2418; A61F 2/90; A61F 2/2436; A61L 27/3625; A61F 2220/0075; A61F 2002/011; A61F 2210/0014; A61F 2250/006
USPC 623/2.11; 606/200

(57) ABSTRACT

A system to limit cerebral ischemia occurring as a consequence of aortic valve replacement includes an aortic valve having proximal and distal ends, a sleeve having proximal and distal ends, the proximal end of the sleeve disposed at the distal end of the aortic valve, and a filter attached to the distal end of the sleeve to receive blood and particles passing from the proximal end of the sleeve to the distal end of the sleeve and to separate the particles from the blood. A related method includes introducing a system into a heart of a patient, the system comprising an aortic valve having proximal and distal ends and a sleeve having a proximal end disposed at the distal end of the valve, implanting the valve in an aortic valve annulus, and filtering blood that has passed through the valve into the sleeve.

### CROSS-REFERENCE TO RELATED APPLICATIONS

This patent is the non-provisional of, and claims the benefit of the filing date of, U.S. Provisional Application No. 61/897,459, filed Oct. 30, 2013. U.S. Provisional Application No. 61/897,459 is hereby incorporated by reference.

### BACKGROUND

This patent is directed to a system and a method to limit cerebral ischemia, and to a system and a method to limit cerebral ischemia during aortic valve replacement, for example.

Aortic valve stenosis—or aortic stenosis—occurs when the aortic valve narrows, preventing the valve from fully opening and obstructing blood flow from the heart. Severe aortic stenosis affects approximately 500,000 people in the United States, of which an estimated 85,000 aortic valve replacement procedures are performed every year. Valve replacement is the most common recommended treatment for aortic stenosis.

A recent development in the area of aortic valve replacement has been the introduction of a transcatheter aortic valve implantation (TAVI) procedure. TAVI is a procedure that permits implanting a new aortic valve (mounted within a stent) through a catheter that can be introduced from the groin, or in some cases, through an aortic or ventricular puncture. The Corevalve system, manufactured by Medtronic Inc. of Minneapolis, Minn., and the Edwards-Sapien system, manufactured by Edwards Lifesciences Inc. of Irvine, Calif., are two examples of valve systems that can be implanted using a retrograde percutaneous procedure. TAVI may permit valve replacement for those persons for whom open surgery is not an option.

This new procedure is not without its risks. A possible severe complication of percutaneous aortic valve replacement is the embolization of valve and atherosclerotic debris into the brain. The debris are small fragments of calcified and diseased aortic valve that break off as the diseased valve is stretched, ruptured and compacted by the new implanted valve.

Following implantation, diffusion weighted magnetic resonance imaging (DWMRI) shows new (silent) cerebral lesions in as many as 80% of patients. The lesions greatly increase the likelihood of vascular dementia in the future. In addition and most significantly, approximately 7% of patients will develop a severe clinical stroke after valve impanation.

As set forth in greater detail below, the present disclosure sets forth a system and method for limiting the release of debris particles into the brain during percutaneous aortic valve replacement.

### SUMMARY

According to an aspect of the present disclosure, a system to limit cerebral ischemia occurring as a consequence of aortic valve replacement includes an aortic valve having a proximal end and a distal end, a sleeve having a proximal end and a distal end, the proximal end of the sleeve disposed at the distal end of the aortic valve, and a filter attached to the distal end of the sleeve to receive blood and particles passing from the proximal end of the sleeve to the distal end of the sleeve and to separate the particles from the blood.

According to another aspect of the present disclosure, a method of limiting cerebral ischemia occurring as a consequence of aortic valve replacement includes introducing a system into a heart of a patient, the system comprising an aortic valve having a proximal end and a distal end and a sleeve having a proximal end disposed at the distal end of the valve, implanting the valve in an aortic valve annulus, and filtering blood that has passed through the valve into the sleeve.

### BRIEF DESCRIPTION OF THE DRAWINGS

It is believed that the disclosure will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings is necessarily to scale.

FIG. 1 is a partial perspective view of an embodiment of a system to limit cerebral ischemia including a valve with an attached sleeve, compacted within a delivery sheath prior to implantation of the valve within the heart;

FIG. 2 is a partial perspective view of the valve during implantation with the delivery sheath being retracted to permit the valve to expand into place;

FIG. 3 is an enlarged, partial perspective view of the valve fully expanded so as to be implanted within the heart with the delivery sheath also being further withdrawn exposing a proximal segment of the sleeve, also expanded;

FIG. 4 is a schematic view of the system in place, with the sleeve extending to a filter used to remove particles from the blood flowing through the sleeve;

FIG. 5 is an enlarged schematic view of the system illustrating the flow of blood through the system;

FIG. 6 is an enlarged partial cross-sectional view of an embodiment of a fastener for attaching the sleeve to the valve in the system illustrated in FIGS. 1-5;

FIG. 7 is an enlarged partial cross-sectional view of the fastener of FIG. 6, with the sutures partially withdrawn;

FIG. 8 is an enlarged partial cross-sectional view of the fastener of FIG. 6, with the sutures fully withdrawn and the sleeve retracted from the valve;

FIG. 9 is an enlarged partial perspective view of another embodiment of a fastener for attaching the sleeve to the valve in the system illustrated in FIGS. 1-5;

FIG. 10 is an enlarged partial perspective view of the fastener of FIG. 9, with the wires partially withdrawn;

FIG. 11 is an enlarged partial perspective view of the fastener of FIG. 9, with the wires fully withdrawn and the sleeve pulled away from the valve; and

FIG. 12 is an enlarged, partial perspective view of a further embodiment in which a valve is fully expanded so as to be implanted within the heart, and a delivery sheath being further withdrawn exposing a proximal segment of a sleeve disposed at the distal end of the valve.

### DETAILED DESCRIPTION OF VARIOUS EMBODIMENTS

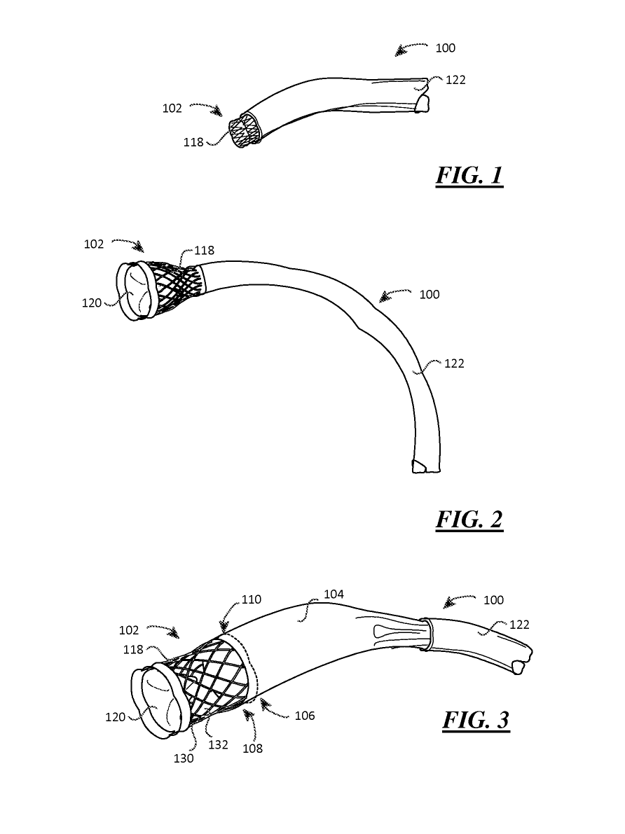

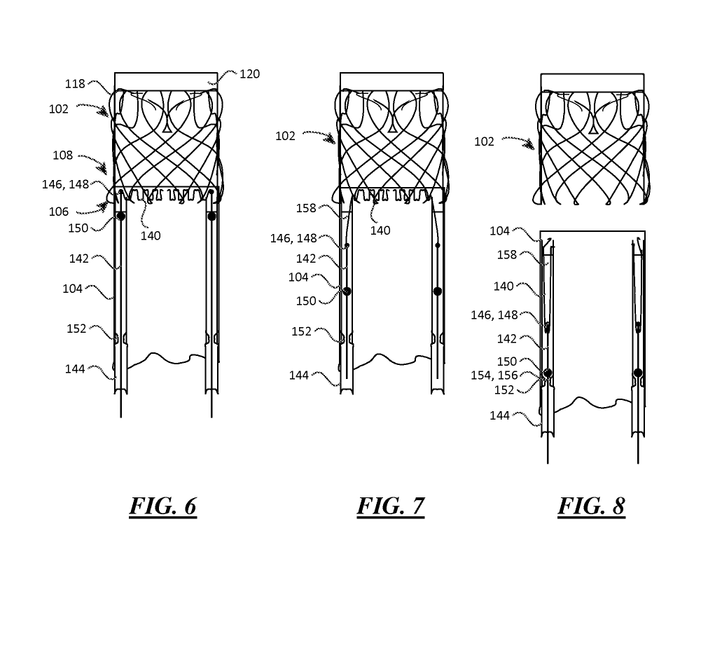

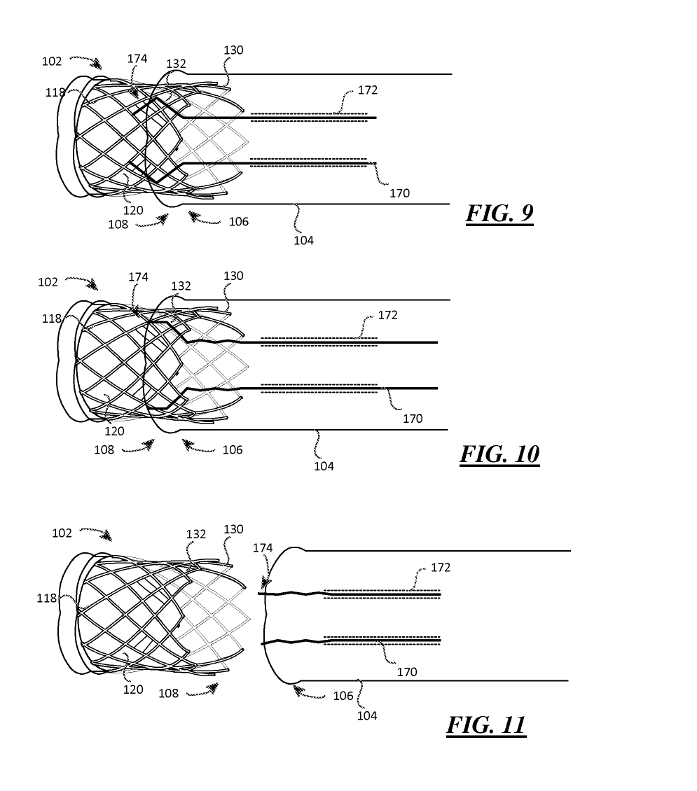

FIGS. 1-5 generally illustrate a system and a method to limit cerebral ischemia during percutaneous aortic valve replacement. Details of a first embodiment of the system are illustrated in FIGS. 6-8, while details of a second embodiment of system are illustrated in FIGS. 9-11. In discussing these embodiments, certain conventions have been adopted regarding the spatial relationships of elements of the disclosed embodiments. For example, elements of the system further from the implantation site have been termed “distal,” while elements of the system closer to the implantation site have been termed “proximal.” This usage is for discussion purposes only, and is not intended to limit the invention set out in the claims.

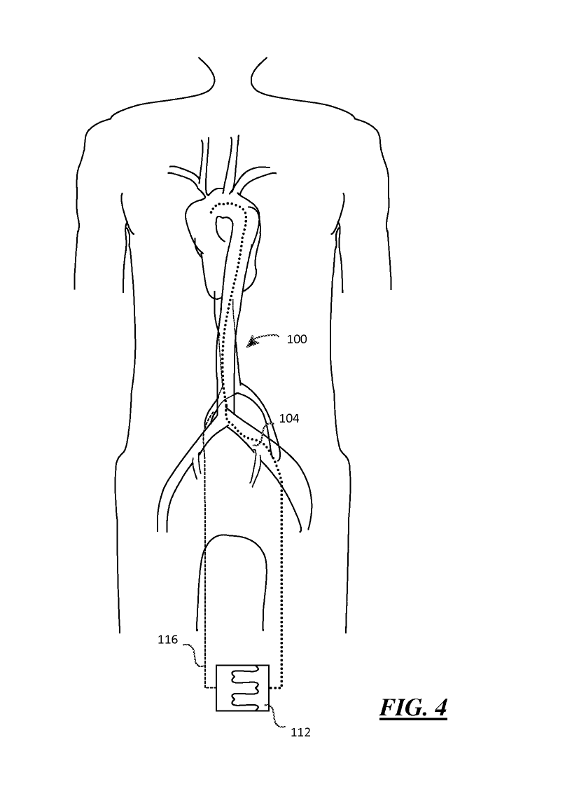

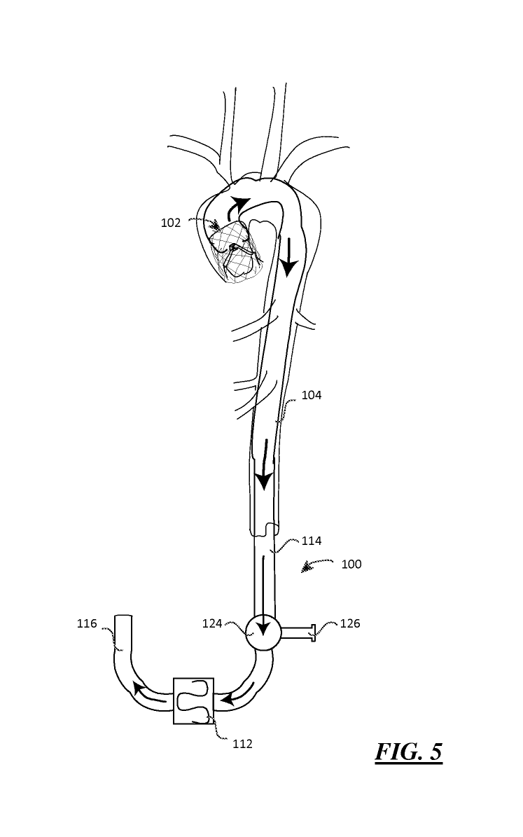

In general terms, the system 100 includes a valve (mounted within a stent) 102, a sleeve 104 that has a proximal (or cephalic) end 106 attached to a distal (or caudal) end 108 of the valve 102, a removable fastener 110 that attaches the proximal end 106 of the sleeve 104 to the distal end 108 of the valve 102, and a filter 112 that is attached to a distal end 114 of the sleeve 104 to receive the blood and particles that enter the proximal end 106 of the sleeve 104 at the distal end 108 of the valve 102 and that pass from the proximal end 106 of the sleeve 104 to the distal end 114 of the sleeve 104. See FIGS. 1-5, and in particular FIGS. 3 and 5. The system 100 may also include a path 116, for example defined by a catheter, to permit the blood that passes through the filter 112 to be returned to the patient, for example via the femoral vein. Thus arranged or configured, the system 100 may create a temporary high-flow arterio-venous fistula.

The system 100 may also include valves and other equipment as will be explained in greater detail below. In fact, it will be recognized that the elements described above may actually be systems, assemblies, or subassemblies in their own right. For example, the valve 102 may include a stent (or valve-stent or stent-frame) 118 to which a valve region formed of an organic biocompatible material, such as an animal tissue valve (e.g., bovine or porcine pericardium) 120 is attached, for example through the use of sutures. In addition, there are other elements, such as a delivery sheath (or introducer) 122 that may be used with the system 100 during the implantation procedure (see FIGS. 1-3) and a valve 124 and sideport 126 for removing equipment that is used for the implantation of the valve 102 (see FIG. 5). To the extent such details are discussed, this will be reserved until after the general use of the system 100 is discussed.

As to the use of the system 100, it will be recognized that the sleeve 104 has been pre-assembled with the valve 102, i.e., the proximal end 106 of the sleeve 104 is attached to the distal end 108 of the valve 102. See FIGS. 1-3. The delivery sheath 122 is disposed about the valve 102 (so as to maintain the stent 118 in a closed or collapsed state or condition) and about the sleeve 104 as well. See FIG. 1.

A guide wire is introduced through the diseased aortic valve and into the heart. Before the valve 102 is implanted, the heart is induced into “rapid pacing.” Rapid pacing of the heart practically arrests any blood flow from the heart, and the heart becomes almost immobile, permitting the precise deployment of the new valve 102. The assembly of the valve 102, sleeve 104, and sheath 122 is then introduced into the heart by advancing the assembly over the guidewire retrograde from the femoral artery to the failing aortic valve, and the valve 102 positioned at the aortic valve annulus. The deployment of the valve 102 may be actuated by inflation of a balloon in the case of a steel or cobalt-chromium stent frame or by self-expansion as the sheath 122 is withdrawn in the case of a nitinol stent frame. Compare FIGS. 1 and 2.

In the case of a stent frame made of nitinol, as the delivery sheath 122 is retracted, the valve 102 is permitted to expand from the collapsed state to an open or deployed state, the shape of the stent 118 ensuring that the valve 102 is positioned and anchored in place within the existing, diseased aortic valve, which is pushed aside. See FIG. 2. As the delivery sheath 122 is further retracted, the proximal end 106 of the sleeve 104 is exposed. See FIG. 3. Further retraction exposes additional portions of the sleeve 104. The rapid pacing used during implantation is interrupted, and blood passes from the valve 102 into the sleeve 104, and through the sleeve 104 and into the filter 112. See FIG. 5. In an embodiment such as is illustrated, the blood that passes through the filter 112 is returned to the patient via the path 116 via the femoral vein. See FIG. 4.

The initial several beats of the heart that occur after rapid pacing is discontinued are believed to bypass a sufficient amount of blood into the sleeve 104 and through the filter 112. This volume of blood contains the debris created by the implantation of the valve 102 in the aortic annulus. Once this sufficient amount of blood has passed through the sleeve 104 and the filter 112, the proximal end 106 of the sleeve 104 may be detached from the distal end 108 of the valve 102. In particular, this may be done by removing the fastener 110 that attaches ends 106, 108. With the fastener 110 removed, the sleeve 104 may be retracted, either separately from the delivery sheath 122 or the sleeve 104 may be kept inside the delivery sheath 122 which is retracted so as to remove both the sheath 122 and the sleeve 104.

Having discussed the system 100 and its method of use in general terms, more specific details of the system 100 and method are discussed below.

As mentioned above, the valve 102 includes the stent 118 and the animal tissue valve 120. See e.g., FIG. 3. The stent 118 may be made of nitinol or cobalt chromium, for example. The stent 118 may have a framework of wire-like structural elements 130 that may define one or more open cells 132. The cells 132 may be advantageously arranged in a diamond cell configuration, although this is not necessary according to all embodiments. The distal end 108 of the valve stent 118 may define a scalloped skirt, so as to better position and anchor the valve 102 in place. The animal tissue valve 120 may be sutured to the stent 118.

The material used for the sleeve 104 may have one or more of the following characteristics. The material preferably should be of sufficient strength to permit attachment to the valve 102 and to accommodate the stresses of introduction along with the remainder of the system 100. In addition, the material preferably should also be tolerant of compression, to permit its introduction compressed or compacted within the delivery sheath 122. The material preferably should be resistant to dilation, and thin to avoid increasing significantly the profile of the system 100. Such material should also be biocompatible, which is generally true of all of the materials described herein. In regard to specific examples of materials that may be used, the sleeve 104 may be made from polytetrafluoroethylene (PTFE), Dacron, or the like. In addition, the sleeve 104 may have one or more (e.g., three) rings of stent at the proximal end 106 that allow it to be deployed against the wall of the aorta together with the valve 102 and thus permit the discharging the entire outflow of the heart into the sleeve 104.

The proximal end 106 of the sleeve 104 may overlap the distal end 108 of the valve 102, and in particular the stent 118, to facilitate attachment to the valve 102 (stent 118). For example, the sleeve 104 may overlap the valve 102 by not more than 2 cm in certain embodiments. Moreover, the proximal end 106 of the sleeve 104 may be funnel-shaped, with the diameter of the sleeve 104 at the proximal end 106 matching the diameter of the distal end 108 of the valve 102 (e.g., 23 mm) and the diameter decreasing progressively (e.g., to 15 mm) for the remaining length of the sleeve 104 to accommodate without infolding within the delivery sheath 122.

The distal end 114 of the sleeve 104 may extend or depend from the end of the delivery sheath 122 and may be connected to a reservoir that defines, at least in part, the filter 112. See FIGS. 4 and 5. As illustrated, the filter 112 also is attached to a cannula (e.g., a sheath) that has been placed percutaneously in the femoral vein to return blood to the patient. The pressure difference between the aorta and the femoral vein may cause the blood to flow rapidly through the sleeve 104, through the filter 112, and into the femoral vein. To permit this high flow rate, the connectors and the filter 112 may have a diameter of not less than 6 mm, and a 10 Fr venous sheath may be used to establish the connection with the femoral vein. By way of example and not by way of limitation, the filter 112 may separate particles that are 120 μm or greater from the blood passing through the filter.

As mentioned above, the sleeve 104 is left in place until a sufficient amount of blood has passed through the system 100. The decision as to how many cardiac beats are necessary to pass a sufficient amount of blood into the sleeve 104 and through the filter 112 may be left to the operator. For example, the operator may permit 5 to 10 beats to occur before removing the sleeve 104. By way of reference, 5 beats would permit approximately 250 cc of blood to pass through the filter 112. It is presently believed that 5 to 10 beats will divert away through the filter 112 most or all of the particles broken free from the implantation of the valve 102.

The sleeve 104 may also be withdrawn through a side port 126 of the system 100 once the valve 102 is deployed and a sufficient amount of blood has been passed through the sleeve 104 and the filter 112.

As mentioned above, the valve 102 is attached to the sleeve 104 through the use of a fastener 104. Like the valve 102, the fastener 110 may be an assembly of one or more individual fastener elements. FIGS. 6-8 illustrate a first embodiment of the fastener 110 including one or more sutures, while FIGS. 9-11 illustrate a second embodiment of the fastener 110 including one or more wires.

First with reference to FIGS. 6-8, an embodiment of the fastener 110 includes, as mentioned above, one or more sutures 140, the one or more sutures 140 being attached at one end to one or more release mechanisms 142, the flexible release mechanisms 142 being disposed in one or more cannulas 144 that may be disposed within and attached to the sleeve 104. As illustrated, there are two sutures 140, two release mechanisms 142 and two cannulas 144, but other embodiments of the fastener may include a greater or lesser number of sutures, release mechanisms and/or cannulas. The illustrated embodiment is thus an exemplary embodiment, and not intended to limit the scope of the claims thereby.

The sutures 142 may be passed or threaded through two thin cannulas or tubes attached to the material of the sleeve 104 and the open cells 130 of the stent 118 to attach the proximal end 106 of the sleeve 104 to the distal end 108 of the valve 102. According to an alternative embodiment, the sutures 140 may be tied with a quick release knot to permit the sutures to be untied and removed. As illustrated, an end 146 of the suture 140 is attached to a proximal end 148 of the release mechanism 142. The suture 140 and the release mechanism 142 may be separate structures, such that the attachment is in the form of a knot, for example, or the release mechanism 142 may be an extension of the suture 140, such that the attachment is seamless and the structures 140, 142 are integral (i.e., formed as a one piece). A distal end of the release mechanism 142 may be manipulated by the operator outside the patient to cause the release mechanism 142 to be retracted or withdrawn. The sutures 140 may be made of a variety of known materials, such as steel, PTFE, nylon, dacron, etc.

The release mechanism 142 is preferably a flexible release mechanism, such as a length of string or cord or an extension of the suture 140. Between the proximal end 148 and the distal end of the release mechanism 142 is a bead-like stop 150 that cooperates with a restriction 152 in the cannula 144. In particular, a surface 154 of the stop 150 abuts a surface 156 of the restriction 152 to limit further motion of the release mechanism 142 relative to the cannula 144. With the cannula 144 fixedly attached to the sleeve 104 such that the cannula 144 is not capable of motion relative to the sleeve 104, limiting the motion of the release mechanism 142 relative to the cannula 144 also limits the motion of the release mechanism 142 relative to the sleeve 104.

The cannula 144 is also preferably flexible to facilitate movement of the system 100 within the circulatory system to permit use in percutaneous valve replacement. For example, the cannulas 144 may be defined by flexible, metallic or plastic minitubes. The proximal end 158 of the cannula 144 is disposed near the proximal end 106 of the sleeve 104, and preferably disposed such that the stop 150 attached to the release mechanism 142 remains within the cannula 144 even before the release mechanism 142 is withdrawn to remove the sutures 140. The distal end of the cannula 144 may extend to the distal end of the release mechanism 142, or may terminate at a point between the proximal end 148 and the distal end of the release mechanism 142.

The embodiment of FIGS. 6-8 is removable in the following fashion, permitting the sleeve 104 to be detached from the valve 102 once the operator has determined that a sufficient amount of blood has passed through the sleeve 104. The operator grasps or otherwise manipulates the distal ends of the release mechanisms 142 to cause the release mechanisms to be withdrawn in the direction of the distal end of the system 100. The movement of the release mechanisms 142 causes the sutures 140 to separate from the sleeve 104 and valve 102 (in particular, the stent-frame 118 of the valve 102). As the release mechanisms 142 are withdrawn within the cannulas 144, the stops 150 move in the direction of the restrictions 152. The distance between the proximal end 148 of each of the release mechanisms 142 and the stop 150 that is attached to (or part of) the release mechanism 142 is such that the surfaces 154, 156 do not abut until the sutures 140 have been separated from the valve 102 (stent 118).

Once the surfaces 154, 156 abut, further pulling on the release mechanisms 142 results in motion of the sleeve 104 away relative to the valve 102. That is, because the abutment of the surfaces 154, 156 limits the motion of the release mechanisms 142 relative to the sleeve 104, as the release mechanisms 142 are withdrawn or retracted in the distal direction, so too is the proximal end 106 of the sleeve 104. The nature of the structure and interaction of the release mechanism 142 and the cannula 144/sleeve 104 may cause this to be referred to as a “ripcord.”

Referring now to FIGS. 9-11, another embodiment of the fastener 110 includes, as mentioned above, one or more wires 170, the wires 170 being disposed in one or more lumens 172 that may be disposed within the wall of the sleeve 104 or that may be defined by one or more cannulas attached to the sleeve 104. As illustrated, there are two wires 172 and two lumens 172, but other embodiments of the fastener may include a greater or lesser number of wires and lumens (e.g., 4 or 6). The illustrate embodiment is thus an exemplary embodiment, and not intended to limit the scope of the claims thereby.

The wires 170 may cooperate with the valve 102, and in particular the stent 118, to attach the sleeve 104 to the valve 102. More specifically, a proximal end 174 of the wire 170 may be disposed in the open cells 132 of the stent 118, or may be passed through the open cells 132 and about the wire-like structural elements 130 of the stent 118. If the former, the wire 170 may be bent in a zig-zag pattern that defines peaks that are disposed in the open cells 132 to engage the stent 118 and to attach the sleeve 104 to the valve 102. If the latter, the wire 170 may appear to be woven into the stent 118 by virtue of being disposed through the open cells 132 and about the wire-like structural elements 130. The wires 170 may also provide a structural effect to the sleeve 104, to maintain the cylindrical shape of the sleeve 104 and to prevent its collapse.

Similar to the release mechanisms 142, the wires 170 are manipulated by the operator to detach the sleeve 104 from the valve 102. That is, by withdrawing or retracting the wires 170 from their engagement with the valve 102, and in particular the stent 118, the sleeve 104 is detached from the valve 102. Once the wires 170 are disengaged, the sleeve 104 may be pulled through the delivery sheath 122 or with the delivery sheath 122.

As will be recognized, the devices according to the present disclosure may have one or more advantages relative to conventional technology, any one or more of which may be present in a particular embodiment in accordance with the features of the present disclosure. For instance, by collecting the blood flowing through the valve immediately after implantation of the valve and filtering the blood to remove blood-borne particles, it is believed that a substantial number (potentially all) of the particles (potential emboli) released during the deployment of the valve will be captured. It is further believed that this will dramatically decrease the risk of stroke caused by emboli released from the aortic valve implantation site. Further, in those embodiments where the system is attached to the valve, this attachment permits simplified introduction of the system to the implantation site with greater control over the placement of the sleeve relative to the valve, all without the addition of steps to the procedure. As such, the method of use of the system is relatively simple and atraumatic. Other advantages not specifically listed herein may also be recognized as well.

Although the preceding text sets forth a detailed description of numerous different embodiments of the invention, it should be understood that the legal scope of the invention is defined by the words of the claims set forth at the end of this patent. The detailed description is to be construed as exemplary only and does not describe every possible embodiment of the invention since describing every possible embodiment would be impractical, if not impossible. Numerous alternative embodiments could be implemented, using either current technology or technology developed after the filing date of this patent, which would still fall within the scope of the claims defining the invention.

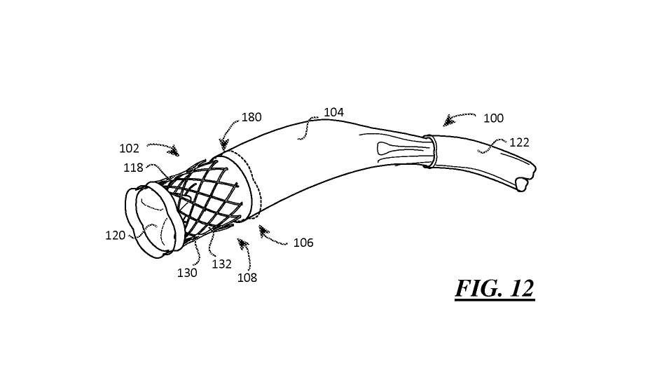

For example, FIG. 12 illustrates an embodiment of the system 100 in which the valve 102 and the sleeve 104 are not attached or joined by a removable fastener. As illustrated, the sleeve 104 has a proximal end 106 disposed at a distal end 108 of the valve 102. According to certain embodiments, the proximal end 106 may abut, or even overlap, the distal end 108 of the valve 102. However, there is no fastener 110 that attaches the proximal end 106 of the sleeve 104 to the distal end 108 of the valve 102.

According to such an embodiment, the system 100 including the valve 102 and the sleeve 104 are compacted inside a sheath 122 to permit proper placement of the sleeve 104 relative to the valve 102. After implantation of the valve 102, the sheath 122 is retracted to expose the sleeve 104. According to certain embodiments, the proximal end 106 of the sleeve 104 may expand synchronously with the expansion of the valve. The expansion of the sleeve 104 may be performed by balloon inflation, or the sleeve 104 may have a self-expanding stent-rings 180 disposed at its proximal end 106. In either event, the proximal end 106 of the sleeve 104 may adapt to the circumference of the wall of the ascending aorta, and once a sufficient volume of blood is shunted through the filter attached to the sleeve 104 (and back into the femoral vein), the sleeve 104 may be removed through a side port.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). Similarly, unless a claim element is defined by reciting the word “means” and a function without the recital of any structure, it is not intended that the scope of any claim element be interpreted based on the application of 35 U.S.C. §112, sixth paragraph. Further, to the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning

We claim:

1. A system to limit cerebral ischemia occurring as a consequence of aortic valve replacement, the system comprising:
an aortic valve having a proximal end and a distal end;
a sleeve having a proximal end and a distal end, the proximal end of the sleeve disposed at the distal end of the aortic valve; and
a filter attached to the distal end of the sleeve to receive blood and particles passing from the proximal end of the sleeve to the distal end of the sleeve and to separate the particles from the blood.

2. The system according to claim 1, further comprising a cannula connected to the filter to return blood from the filter to the patient.

3. The system according to claim 1, further comprising a removable fastener that attaches the proximal end of the sleeve to the distal end of the valve.

4. The system according to claim 3, wherein the removable fastener comprises at least one suture that attaches the proximal end of the sleeve to the distal end of the valve.

5. The system according to claim 4, wherein the removable fastener comprises at least one flexible release mechanism having a proximal end attached to the at least one suture and a distal end.

6. The system according to claim 5, wherein the at least one flexible release mechanism is an extension of the at least one suture.

7. The system according to claim 5, wherein the removable fastener comprises at least one cannula fixedly attached to the sleeve,
the at least one cannula having proximal and distal ends,
the at least one flexible release mechanism disposed within the at least one cannula, and
the at least one flexible release mechanism including a stop disposed between the proximal and distal ends of the at least one flexible release mechanism and the at least one cannula including at least one restriction disposed between the proximal and distal ends, the stop abutting the restriction to limit the relative movement of the release mechanism to the cannula and the attached sleeve.

8. The system according to claim 2, wherein the valve comprises a stent having at least one open cell and the removable fastener comprises at least one wire having a proximal end cooperating with the at least one open cell to attach the fastener to the stent and a distal end.

9. The system according to claim 8, wherein the stent includes wire-like structural elements that define the at least one open cell, the proximal end of the wire disposed through the at least one open cell and about the wire-like structural elements.

10. The system according to claim 1, further comprising a delivery sheath, the valve and at least a portion of the sleeve disposed within the delivery sheath prior to implantation of the valve.

11. The system according to claim 1, wherein the filter is capable of removing particles 120 μm and larger.

12. The system according to claim 1, wherein the valve comprises a stent and an animal valve disposed within the stent.

13. A method of limiting cerebral ischemia occurring as a consequence of aortic valve replacement, the method comprising:
introducing a system into a heart of a patient, the system comprising an aortic valve having a proximal end and a distal end and a sleeve having a proximal end disposed at the distal end of the valve;
implanting the valve in an aortic valve annulus; and
filtering blood that has passed through the valve into the sleeve.

14. The method according to claim 13, further comprising rapid pacing the heart of the patient prior to introducing the system and interrupting rapid pacing prior to filtering blood that has passes through the valve into the sleeve.

15. The method according to claim 13, wherein filtering blood that has passed through the valve into the sleeve comprises filtering blood that has passed through the valve into the sleeve for 5 to 10 heart beats.

16. The method according to claim 13, further comprising returning blood that has passed through the valve into the sleeve after filtering the blood that has passed through the valve into the sleeve.

17. The method according to claim 13, further comprising detaching the sleeve from the valve.

18. The method according to claim 17, wherein detaching the sleeve from the valve comprises removing one or more sutures attaching the sleeve to the valve.

19. The method according to claim 17, wherein detaching the sleeve from the valve comprises removing one or more wires attaching the sleeve to the valve.

20. A filter assembly for trapping debris caused by implantation of an artificial aortic valve, comprising:
a sleeve having a proximal end and a distal end, the proximal end of the sleeve being connectable to a self-expanding stent frame of a valve, the sleeve tapering from a first diameter at the proximal end thereof, the first diameter equaling a diameter of a distal end of the valve, inwardly to a second diameter in a direction toward the distal end of the sleeve;
the valve having a proximal end and a distal end, the valve including a valve region comprising an organic biocompatible material with a distal end defining a scalloped skirt, and the self-expanding stent frame attached to the valve region, the stent frame defined by a framework of wire-like structural elements defining a plurality of open cells.
